(19) United States
(12) Patent Application Publication
(10) Pub. No.: US 2024/0261344 A1
(43) Pub. Date: Aug. 8, 2024

(54) Method of alleviating symptoms of depression and anxiety by use of a novel Bacillus coagulans strain

(71) Applicant: Unique Biotech Limited, Hyderabad (IN)

(72) Inventors: Ratna Sudha Madempudi, Hyderabad (IN); Manoj P. Dandekar, Hyderabad (IN); Manisurya Kumar Palepu, Hyderabad (IN); Srilakshmi Satya Satti, Hyderabad (IN)

(21) Appl. No.: 18/609,012

(22) Filed: Mar. 19, 2024

(30) Foreign Application Priority Data
Jan. 12, 2023 (IN) 202341018674

(51) Int. Cl. A61K 35/742; A61P 25/22; A61P 25/24

(52) U.S. Cl. CPC A61K 35/742; A61P 25/22; A61P 25/24

(57) ABSTRACT

The current invention more specifically relates to methods of amelioration of symptoms of anxiety and depression by using a probiotic formulation comprising Bacillus coagulans. The probiotic formulation used for the amelioration of depression and anxiety symptoms may comprise a novel strain of Bacillus coagulans (Unique IS-2) (MTCC 5260, ATCC PTA-11748), which is used for treating, preventing or ameliorating at least one symptom of anxiety, stress, depression or a depressive disorder.

The current invention discloses a method for treating, preventing or ameliorating at least one symptom of depression or a depressive disorder, the method comprising the step of administering to a subject an effective dosage regimen of a probiotic formulation comprising Bacillus coagulans.

### FIELD OF INVENTION

The current invention relates to the field of probiotic formulation for alleviating symptoms of depression and anxiety. The current invention more specifically relates to methods and compositions comprising Bacillus coagulans that can be used for alleviating symptoms of depression in humans. The strain of Bacillus coagulans is more specifically Bacillus coagulans Unique IS-2 that can be used for alleviating symptoms of depression in humans.

### BACKGROUND

According to World Health Organization, >300 million people are affected with depression worldwide; and many others remain undiagnosed. A marked rise in depressive cases has also been noted after the COVID-19 pandemic compared to the pre-pandemic years. Although a wide range of antidepressant agents are available, treatment-resistant depression, treatment-associated complications, and other psychiatric comorbidities with depression are not satisfactorily addressed yet. Thus, there are unmet needs for newer therapeutic approaches which have fewer side effects and increased efficacy. Many of the known neuropathologies of depression, such as monoamine neurotransmitters depletion, hyperactivation of the hypothalamic-pituitary-adrenal (HPA) axis, and immune imbalances, are interconnected with changes in gut microbiome. The crosstalk between the intestine and the brain referred to as the microbiome gut-brain axis, has also been increasingly reported in CNS disorders. Recent findings have reported an incidence of gut dysbiosis (i.e., imbalance in the microbial community) in depression, which may impact the integrity of the intestinal wall, increase the colonization of pathogenic bacteria, and also modulate the production of neurotransmitters, proinflammatory cytokines, neurotrophic factors, hormones, neuropeptides, and tryptophan metabolism (Ref 1, Capuco et al).

Several studies have demonstrated the importance of the gut microbiome in the neuropathology and management of depression. An altered gut microbiome signature has also been noted in depressive patients (Ref 2, Zheng et al), indicating the imbalance between beneficial and harmful bacterial colonies in the intestine of the host. Zheng et al (Ref 2) describes the causative association between major depressive disorder (MDD) and gut microbiota. Major depressive disorder (MDD) is a debilitating mental disorder affecting up to 15% of general population and accounting for 12.3% of the global burden of disease. MDD has been shown to be associated with disturbances in peripheral and central metabolites, which are metabolic by products of gut microbiota. Some clinical studies have also reported disturbances in the gut microbiota compositions in limited samples of heterogeneous depressed subjects with similar findings observed in animal models of depression (Ref 2, Zheng et al). There is increasing amount of scientific evidence demonstrating that gut microbiota can greatly influence all aspects of physiology. Emerging evidence also suggests that gut microbiota can influence brain function and behaviour through the ‘microbiota-gut-brain axis’ (Ref 3, Jiang et al). Thus, the remodelling of commensal gut microbiota may become one of the treatment strategies to more effectively relieve depressive symptoms.

The profile of the gut microbiome can be modified using the administration of prebiotics, probiotics, postbiotics, and fecal microbiota transplantation (FMT). Interestingly, transplantation of depressive patients' fecal microbiota into germ-free (GF) mice showed depressive-like behavior. Therefore, the introduction of FMT as a beneficial microbial gut community from a healthy donor to depressive patients is also being investigated by several laboratories. In fact, administration of FMT was reported to improve depressive symptoms after four weeks of treatment in two patients with major depressive disorder. In addition, several preclinical and clinical studies have also shown mood-elevating effects of probiotics via the microbiome-gut-brain axis

Bacillus coagulans is a spore-forming, lactic acid-producing, and facultative non-pathogenic anaerobic bacterium, which can survive in the extreme microenvironment of the intestine and germinate in the small intestine (Ref 4, Keller et al). This bacterium is labelled as a “Generally Recognized as Safe (GRAS)” bacteria for human and animal consumption. Bacillus coagulans strain is also known to regulate the host symbiotic microbiota, rescind the growth of pathogenic bacteria, and improve the functional activity of immune cells (Ref 5, Cao et al).

The commercial probiotic strain, Bacillus coagulans Unique IS-2 (MTCC 5260, ATCC PTA-11748), has also been found to be efficacious in the treatment of diarrhoea, IBS, IBD, constipation, bacterial vaginosis, dental caries and hypercholesterolemia

The current invention encompasses a method of ameliorating symptoms associated with depression, and anxiety, by treating subjects with Bacillus coagulans Unique IS-2 for at least six weeks, with an effective dose regimen. The current invention encompasses the use of a probiotic formulation comprising Bacillus coagulans Unique IS-2 in managing anxiety and depression.

### SUMMARY

One embodiment of this invention is a method of treating, preventing or ameliorating at least one symptom of anxiety or depression in a subject, the method comprising the step of administering to the subject an effective dosage of a probiotic formulation comprising the bacterial strain Bacillus coagulans.

In one embodiment, the B. coagulans in the probiotic formulation used in method disclosed herein is the strain Bacillus coagulans Unique IS-2 (MTCC 5260; ATCC PTA-11748).

In one embodiment, the administration of the effective dosage regimen of the probiotic formulation does not affect body weight or locomotor activity of the subject.

In one embodiment, the effective dosage regimen of the probiotic formulation comprising B. coagulans is 1 dose a day administered every day for 6 weeks.

In one embodiment, the effective dosage regimen of the probiotic formulation is 1-2 doses a day administered every day for six weeks.

In one embodiment, each dose of the probiotic formulation comprises 2 billion cfu of Bacillus coagulans.

In one embodiment, each dose of the probiotic formulation comprises 1-3 billion cfu of Bacillus coagulans.

In one embodiment, the probiotic formulation is administered to the patient as an adjunct to standard medical treatment (SMT) for anxiety or depression.

In one embodiment, the probiotic formulation is administered orally to the patient.

In one embodiment, the probiotic formulation is administered to the subject in the form of a food product, a dietary supplement or a pharmaceutically acceptable composition.

In one embodiment, the probiotic formulation alleviates anxiety and depression symptoms by increasing gut eubiosis in the patient.

In one embodiment, the administration of an effective dosage of the probiotic formulation comprising B. coagulans leads to amelioration of one or more symptoms associated with anxiety and/or stress, such as gastrointestinal disorders (IBS), back pain, chest pain, shortness of breath, heart palpitations, problems with sleep or appetite, and fatigue.

In one embodiment, administration of effective dosage of the probiotic formulation comprising B. coagulans IS2 leads to amelioration of one or more of symptoms associated with anxiety and/or depression such as irritability, mood swings, depressed mood, disturbed sleep, listlessness, cognitive changes, short term memory loss, anxiousness, restlessness, tension, and anhedonia.

In one embodiment, administration of an effective dosage of the probiotic formulation comprising B. coagulans leads to decrease of the depression symptom of anhedonia in subjects treated with the formulation.

In one embodiment, the administration of the effective dosage regimen of probiotic formulation alleviates anxiety and depression symptoms by modulating levels of tryptophan metabolites.

In one embodiment, the administration of the effective dosage regimen of probiotic formulation ameliorates the symptoms associated with anxiety and/or depression by restoring the elevated levels of L-tryptophan, L-kynurenine, kynurenic acid, and 3-hydroxyanthranilic acid in the circulatory system of the subject.

In one embodiment, the administration of the effective dosage regimen of probiotic formulation ameliorates the symptoms associated with anxiety and/or depression by reversing the accumulation of neurotoxic metabolites associated with anxiety or depression in the subject. In one embodiment, the neurotoxic metabolite is 3-hydroxy anthranilic acid.

In one embodiment, the administration of the effective dosage regimen of probiotic formulation ameliorates the symptoms associated with anxiety and/or depression by reversing the decrease in levels of short chain fatty acids associated with anxiety or depression in the subject. In one embodiment, the short chain fatty acids are acetate, propionate and/or butyrate.

In one embodiment, the administration of an effective dosage regimen of the probiotic formulation to the subject ameliorates the symptoms associated with anxiety and/or depression by decreasing the levels of proinflammatory cytokines in the subject.

In one embodiment, administration of an effective dosage regimen of the probiotic formulation ameliorates the symptoms associated with anxiety and/or depression by reversing the elevated expression levels of IL-1β, TNF-α and C-reactive protein associated with anxiety and depression in the subject.

In one embodiment, the administration of an effective dosage regimen of the probiotic formulation to the subject ameliorates the symptoms associated with anxiety and/or depression by increasing in the number of goblet cells and improved crypt-to-villi ratio in the colon of the subject.

In one embodiment, administration of an effective dosage regimen of the probiotic formulation ameliorates the symptoms associated with anxiety and/or depression by reversing the decrease in BDNF levels in subjects showing symptoms of anxiety and/or depression.

In one embodiment, administration of an effective dosage regimen of the probiotic formulation ameliorates the symptoms associated with anxiety and/or depression by reversing the decrease in Firmicutes to Bacteroidetes ratio in subjects showing symptoms of depression and/or anxiety.

### BRIEF DESCRIPTION OF FIGURES

FIG. 1 illustrates the study design showing that early- and late-life stress was employed via maternal separation (MS) from postnatal (PND) day 2 through PND 14 and chronic unpredictable mild stress (CUMS) at the age of 12th week for 2 weeks. Behavioural assessments were done 6 weeks after Bacillus coagulans treatment.

FIG. 2 shows the Effect of Bacillus coagulans probiotic formulation on sucrose consumption in SPT (A) and immobility time in FST (B), in MS+CUMS exposed male and female rats. The data (each bar) is represented as mean±S.E.M (n=6-8).

FIG. 3. Shows the effect of Bacillus coagulans probiotic formulation on anxiety-like phenotype in MS+CUMS exposed male and female rats. The graphs showing percent open arm entries (A), percent time spent in open arms (B), and total closed arm entries (C). Each bar is represented as mean±S.E.M.

FIG. 4. Effect of Bacillus coagulans probiotic formulation treatment on levels of TNF α and CRP in the frontal cortex (A and C) and hippocampus (B and D) of MS+CUMS exposed male and female rats. The data were analyzed by one-way ANOVA, followed by post-hoc Dunnett's multiple comparisons test. The sex-specific correlation was assessed using two-way ANOVA, followed by Bonferroni's multiple comparisons test.

FIG. 5 shows the Effect of Bacillus coagulans Unique IS-2 treatment on IL-1ß cytokine in the frontal cortex of MS+CUMS exposed male and female rats. The data were analyzed by one-way ANOVA, followed by post-hoc Dunnett's multiple comparisons test. The sex-specific correlation was assessed using two-way ANOVA, followed by Bonferroni's multiple comparison's test.

FIG. 6. Effect of Bacillus coagulans Unique IS-2 on the concentration of dopamine (DA) (A), noradrenaline (also called Norepinephrine) (NA) (B), and 5-HT (5-hydroxytryptamine or Serotonin) (C) in the frontal cortex of MS+CUMS subjected male and female rats.

FIG. 7. Effect of Bacillus coagulans Unique IS-2 on the astrocyte activation (GFAP), tight junction protein (zona occludens) and neurogenesis (doublecortin) in the hippocampus of MS+CUMS exposed male and female rats. The quantification of GFAP (A), zona occludens (B) and doublecortin (C) protein levels was done using ImageJ analysis. The protein levels were normalized with β-actin, and represented in the bar graph as mean±S.E.M. The representative immunoblotting images of each protein are presented (D).

FIG. 8. Effect of Bacillus coagulans Unique IS-2 treatment on the abundance of intestinal bacterial population in fecal samples of MS+CUMS exposed male and female rats.

FIG. 9. Shows Effect of Bacillus coagulans Unique IS-2 treatment on tryptophan metabolites in MS+CUMS exposed male and female rats. The plasma levels of (A) L-Tryptophan, (B) L-Kynurenine, (C) Kynurenic acid, and (D) Hydroxyanthranilic acid metabolites were quantified. Percentage with respect to vehicle control are calculated and represented as mean±S.E.M. for 6 rats.

FIG. 10. Effect of Bacillus coagulans Unique IS-2 (probiotic) on intestinal tight junction protein, occludin in MS+CUMS exposed rats. Photomicrographs showing occludin-immunoreactivity in the distal part of colon sections of the vehicle control (male rats) [A], vehicle+stress (MS+CUMS) in male rats [B], probiotic+stress (MS+CUMS) in male rats [C], vehicle control (female rats) [D], vehicle+stress (MS+CUMS) in female rats [E], and probiotic+stress (MS+CUMS) in female rats [F]. The mean occludin intensity is represented in the bar graph [G] as mean±S.E.M. Scale bar=100 μm.

### DETAILED DESCRIPTION

Depression and anxiety are two of the most prevalent mental health disorders. The main clinical features of depression are loss of interest, often accompanied by guilt, hopelessness, loss of appetite and insomnia. Anxiety is an emotional state where the main characteristics are tension, worry, fear, and physical changes such as palpitations, tremors, gastrointestinal tract, respiratory and circulatory disorders without obvious objective causes. The World Health Organization (WHO) estimated that the prevalence of depression in the global population was as high as 4.4% in 2015, and the prevalence of anxiety disorders was estimated to be 3.6%. The Covid pandemic has exacerbated the prevalence of depression and anxiety. Moreover, depression and anxiety frequently co-occur.

Many instances of anxiety and depression manifest in physical symptoms, especially in primary care settings, occurrences manifest themselves in several ways and have different clinical courses. The prevalence of depressive and anxiety disorders in primary care settings is high. Depression and anxiety disorders contribute to many primary care/physician visits with undiagnosed physical symptoms that may be associated with depression and/or anxiety. If unrecognized and undiagnosed, depression and anxiety disorders contribute to high medical utilization in the primary care setting due to complaints for somatic rather than psychological problems. Back pain, chest pain, shortness of breath, heart palpitations, problems with sleep or appetite, and fatigue are among the most frequently presenting symptoms. Excessive anxiety is also associated with many negative health consequences, such as increased risk of coronary heart disease, sleep disorders.

### Definitions

As used herein the term “and/or” encompasses “both” and “either/or”. As used herein term “or” may also refer to existence of symptoms of either anxiety or depression or of both.

As used herein, “treating and preventing depression, anxiety or a depressive or anxiety-related disorder” includes, the amelioration or alleviation, at least in part, of one or more symptoms of the depression, depressive disorder, anxiety or anxiety-related disorder. For example, symptoms of depression, anxiety and related disorders that may be inhibited, a or alleviated include irritability, mood swings, depressed mood, disturbed sleep, listlessness, cognitive changes, short term memory loss, anxiousness, restlessness, tension, anhedonia, poor self-esteem, suicidal thoughts or suicidal tendencies.

Further, “treating or preventing” includes preventing the development of depression, anxiety or a depressive or anxiety-related disorder in a subject that may be predisposed to such a condition or may display one or more symptoms of such a condition but has not yet been diagnosed with the condition. “Treating or preventing” also include preventing the onset of a depressive episode or anxiety episode in a subject, or preventing the appearance of symptoms.

As used herein “effective dosage regimen” refers to the specific way a therapeutic drug is to be taken, including formulation, route of administration, dose, dosing interval, and treatment duration, so as to have the desired effect.

In the current invention “effective dosage regimen” of the probiotic formulation has the desired effect of preventing, treating and/or ameliorating anxiety and/or stress symptoms.

As used herein, the term “dose” refers to the measured and specific amount of a therapeutic composition, administered at one time. The number, and frequency of doses given over a specified period of time or prescribed intervals makes up the dosage regimen.

The term “conventional pharmaceuticals or compounds” or “conventional depression treatment therapy” or “Standard Medical Treatment” as used herein in the context of “other conventional pharmaceuticals or compounds used to treat depression or anxiety/stress disorder/symptoms” and refers to those pharmaceuticals or compounds that persons of skill in the art (including but not limited to physicians) conventionally use to treat the “condition/disorder/symptom” or “behavioural abnormality” associated with depression or anxiety/stress disorder.

As used herein, the term “effective amount” refers to the amount of a compound sufficient to effect beneficial or desired results. An effective amount can be administered in one or more administrations, applications or dosages and is not intended to be limited to a particular formulation or administration route.

“Therapeutically effective amount” includes an amount of a compound of the invention that is effective when administered alone or in combination to treat or prevent the onset of the disease condition or disorder. For a combination of compounds, each with a therapeutic effect, “therapeutically effective amount” of the combination of compounds is an amount of the combination of compounds claimed that is effective to treat the disease condition or disorder. The combination of compounds can be additive and is preferably a synergistic combination.

The term “treatment-resistant depression” as used herein refers to any form of depression or related disorder that in any one or more given individuals has not responded to at least one adequate trial with antidepressant therapy, or that responds to a lesser extent or displays a diminished or reduced response to said treatment when compared to depression or related disorder in a subject that displays no resistance to said treatment. Thus, “treatment-resistant depression” may indicate complete or partial resistance to treatment.

Thus, as used in the context of the current specification, an individual suffering from depression or a related disorder that does not respond to a particular treatment may be referred to a ‘non-responder’ with respect to that treatment, and an individual suffering from depression or a related disorder that does not respond fully to a particular treatment (i.e. shows some level of resistance to treatment such that response is diminished when compared to the response of an individual suffering from depression or a related disorder that does not display resistance to said treatment) may be referred to a ‘suboptimal-responder’ with respect to that treatment (displays ‘suboptimal treatment response’). The depression suffered by the individual may be referred to as treatment-resistant depression with respect to that treatment.

The term “Anhedonia” as used herein refers to the reduced ability to experience pleasure. It may be social or physical anhedonia. Anhedonia is considered as a core feature of major depressive disorder.

Depression is an affective disorder with a heterogeneous group of symptoms.

The exact pathophysiological mechanisms for effectiveness of different types of treatments are not understood completely so far. Some of the factors that are being researched are dysfunctions on the hypothalamic-pituitary axis, neurotransmitters, vagus nerve, short-chain fatty acid metabolites, tryptophan, inflammatory factors and the brain-gut-microbiota axis. Many studies have shown that not only do stress-related disorders bring changes in the composition of the intestinal microbiota through the hypothalamic-pituitary-adrenal axis but the intestine also has an effect on the central nervous system through vagal stimulation, intestinal permeability and the release of inflammatory and anti-inflammatory compounds and changes in circulating agents in the blood. Many studies have also focused on the leaky gut syndrome contributing to neuropathological disorders.

The main role of the intestinal barrier is in the digestion and absorption of nutrients; but it also controls the transport of antigens from the intestinal lumen into the submucosa, and maintains the balance between tolerance and the immune response to antigens that causes inflammation. The integrity of the intestinal barrier can be affected by diet, dysbiosis of the intestinal microbiota or by other factors. These have the potential to cause immune activation by translocation of microbial antigens and metabolites.

### (Ref 6, Iordache et al).

Many of the current research efforts are focused on the identification of specific microbial signatures for many inflammatory and metabolic diseases. more particularly for those associated with obesity, type 2 diabetes, and cardiovascular diseases. The relative abundance of Firmicutes, Bacteroidetes, Actinobacteria, and Proteobacteria has been reported between depressive people and healthy controls. The higher abundance of Firmicutes and Actinobacteria, and lower abundance of Bacteroidetes have been reported in depression patients (Ref 7, Liu et al). Moreover, there is an association between irritable bowel syndrome (IBS) and incidence of depression and anxiety (Ref 6 & 8, Iordache et al & Liu et al). IBS is the most prevalent functional gastrointestinal disorder, affecting 10% of the population. Comorbidity of IBS with psychological distress is common. A higher ratio of Firmicutes/Bacteroidetes, has also been reported, leading to lower diversity in gut microflora. IBS patients had a high abundance of Bacteroides in mucosal microbiota, which was reduced after probiotic treatment. Jiang et al (Ref 3) reported that there is relative greater abundance of, Bacteroidetes, Actinobacteria, and Proteobacteria in depressive people compared to healthy controls. The higher abundance of Firmicutes and Actinobacteria, and lower abundance of Bacteroidetes have been reported in depression patients (Ref 3, Jiang et al). Also reported a lower abundance of Firmicutes in people with depression (Ref 3, Jiang et al). Thus, patients with depression have also been found to have microbiota dysbiosis. Therefore, microbiota balance might play an important role in supporting emotional well-being.

Depression or a depressive disorder is typically diagnosed only when the symptoms reach a threshold and last at least two weeks. A number of methods, tools and techniques that are well known to those skilled in the art are used for diagnosing depression, anxiety and depressive or anxiety-related disorders, and for assessing the status or severity of the depression or anxiety, and their symptoms over time, for monitoring the change in status or severity of such conditions or symptoms over a period of time, including in response to treatment or therapy.

Such methods and techniques for diagnosing, assessing and monitoring depression, anxiety and depressive or anxiety-related disorders may comprise clinician assessment, self-assessment or self-reporting questionnaires, and clinician-completed reports or questionnaires, in addition to biochemical measurements. A variety of clinical measures of symptoms and mood are well-known to those skilled in the art.

Any of such method(s) or technique(s) in diagnosing depression, anxiety or a depressive or anxiety-related disorder and for assessing or monitoring of the symptoms may be used, for example, for an initial determination of the suitability of an individual to be treated in accordance with the present disclosure or a determination of the efficacy of a treatment in accordance with the present disclosure in an individual.

Exemplary self-assessment or self-report questionnaires include, but are not limited to the Depression and Anxiety Stress Scale (DASS), the Outcome Quiestionnaire-45 (OQ45), Quality of Life in Depression Scale (QLDS, including a Quality of Life (QoL) score), the Beck Depression Inventory (BDI), the Warwick-Edinburgh Mental Well-Being Scale (WBS), the Mini International Neuropsychiatric Interview (MINI), the Structured Clinical Interview for DSM Disorders (SCID) and the Patient Health Questionnaire (PHQ, such as PHQ-9 and PHQ-2). Exemplary clinician-completed reports or questionnaires include, but are not limited to, the Hamilton Depression Rating Scale (HAM-D) and the Raskin Depression rating Scale. Biochemical measurements that may be employed include, but are not limited to, whole blood serotonin levels.

The application of chronic stress predominately alters the neural activity in the frontal cortex and the hippocampus. The levels of important neurotransmitters such as dopamine (DA), serotonin (5-HT), and noradrenaline (NA) are observed to be changed in the brain of individuals affected by MDD or anxiety. The levels of Brain derived neurotrophic factor (BDNF) and proinflammatory cytokines are also altered. The expression of neurogenesis marker doublecortin (DCX), a precursor protein expressed in immature neurons is usually affected following stressful stimuli. The occurrence of gut dysbiosis has also been linked with the appearance of pathogenic bacteria and increased expression of proinflammatory cytokines. This physiological event promotes the tryptophan metabolism by activating indoleamine-2,3-dioxygenase to the kynurenic acid pathway (Ref 9, Chen et al). have reported that depressive behaviors induced by cytokines are related to the activation of the kynurenine pathway (KP). Studies based on animal models of depressive symptoms have reported that indoleamine 2, 3-dioxygenase (IDO) is likely to be a key metabolic enzyme in the kynurenine pathway. Moreover, increased levels of TNF-α and IL-6 activate the enzymes of the kynurenine pathway, which convert the tryptophane to kynurenine. Also, stress and anxiety can lead to a significant decrease in the villi/crypt ratio and the number of goblet cells.

Thus, there is a lot of evidence supporting the theory that the microbiome gut-brain axis and compromised intestinal integrity plays a crucial role in the pathophysiology of depression and anxiety.

### Embodiments

One embodiment of this invention is a method of treating, preventing, and/or ameliorating at least one symptom of anxiety or depression in a subject, the method comprising the step of administering to the subject an effective dosage regimen of a probiotic formulation comprising the bacterial strain Bacillus coagulans.

In one embodiment, the B. coagulans in the probiotic formulation is the strain Bacillus coagulans Unique IS-2 (MTCC 5260).

In one embodiment, the effective dosage regimen of the probiotic formulation comprising B. coagulans is 1-2 doses a day administered every day for 4-8 weeks.

In one embodiment, the effective dosage regimen of the probiotic formulation is 1-2 doses a day administered every day for six weeks.

In one embodiment, each dose of the probiotic formulation comprises 1-10 billion cfu of Bacillus coagulans.

In one embodiment, each dose of the probiotic formulation comprises 1-3 billion cfu of Bacillus coagulans.

In one embodiment, the probiotic formulation is administered to the patient as an adjunct to standard medical treatment (SMT) for anxiety or depression.

In one embodiment, the probiotic formulation is administered orally to the patient.

In one embodiment, the probiotic formulation is administered to the subject in the form of a food product, a dietary supplement or a pharmaceutically acceptable composition.

In one embodiment, the probiotic formulation alleviates anxiety and depression symptoms by increasing gut eubiosis in the patient.

In one embodiment, the administration of an effective dosage of the probiotic formulation comprising B. coagulans leads to amelioration of one or more symptoms associated with anxiety, such as gastrointestinal disorders (IBS), back pain, chest pain, shortness of breath, heart palpitations, problems with sleep or appetite, and fatigue.

In one embodiment, administration of effective dosage of the probiotic formulation comprising B. coagulans leads to amelioration of one or more of symptoms associated with anxiety or depression such as irritability, mood swings, depressed mood, disturbed sleep, listlessness, cognitive changes, short term memory loss, anxiousness, restlessness, tension, and anhedonia.

In one embodiment, administration of an effective dosage of the probiotic formulation comprising B. coagulans leads to decrease of the depression symptom of anhedonia in subjects treated with the formulation.

In one embodiment, the administration of the effective dosage regimen of probiotic formulation alleviates anxiety and depression symptoms by modulating levels of tryptophan metabolites.

In one embodiment, treatment with the probiotic formulation comprising B. coagulans leads to normalization of the increased plasma levels of L-tryptophan, L-kynurenine, kynurenic acid, and 3-hydroxyanthranilic acid found associated with depression and anxiety.

In one embodiment, the administration of the effective dosage regimen of probiotic formulation ameliorates the symptoms associated with anxiety and/or depression by reversing the accumulation of neurotoxic metabolites associated with anxiety or depression in the subject. In one embodiment, the neurotoxic metabolite is 3-hydroxy anthranilic acid.

In one embodiment, the administration of the effective dosage regimen of probiotic formulation ameliorates the symptoms associated with anxiety and/or depression by reversing the decrease in levels of short chain fatty acids associated with anxiety or depression in the subject. In one embodiment, the short chain fatty acids are acetate, propionate and/or butyrate.

In one embodiment, the administration of an effective dosage regimen of the probiotic formulation ameliorates the symptoms associated with anxiety and/or depression by decreasing levels of proinflammatory cytokines in the subject.

In one embodiment, administration of an effective dosage regimen of the probiotic formulation ameliorates the symptoms associated with anxiety and/or depression by reversing the elevated expression levels of IL-1β, TNF-α and C-reactive protein (CRP) associated with anxiety and depression in the subject.

In one embodiment, the administration of an effective dosage regimen of the probiotic formulation to the subject ameliorates the symptoms associated with anxiety and/or depression by increasing in the number of goblet cells and improved crypt-to-villi ratio in the colon of the subject.

In one embodiment, administration of an effective dosage regimen of the probiotic formulation ameliorates the symptoms associated with anxiety and/or depression by reversing the decrease in BDNF levels in subjects showing symptoms of anxiety and/or depression.

In one embodiment, administration of an effective dosage regimen of the probiotic formulation ameliorates the symptoms associated with anxiety and/or depression by reversing the decrease in Firmicutes to Bacteroidetes ratio in subjects showing symptoms of depression and/or anxiety.

One embodiment of the current invention is a probiotic formulation comprising a Bacillus coagulans Unique IS-2 strain (MTCC 5260, ATCC PTA-11748), which is used for treating, preventing or ameliorating at least one symptom of anxiety, stress, depression or a depressive disorder.

One embodiment of the current invention is a method for treating, preventing or ameliorating at least one symptom of depression or a depressive disorder, the method comprising the step of administering to a subject an effective dose of Bacillus coagulans Unique IS-2.

In one embodiment, the Bacillus coagulans Unique IS-2 probiotic formulation is administered at a dosage of 1-10 billion cfu, administered for 4-8 weeks, given once or twice a day.

In one embodiment, stress-generated neurobehavioral changes and symptoms are alleviated by administration of the probiotic formulation comprising Bacillus coagulans.

In one embodiment, the B. coagulans Unique IS-2 formulation is administered for a period of at least four weeks to the subject/patient. In one embodiment, the B. coagulans Unique IS-2 formulation is administered for a period of at least six weeks to the subject/patient.

In one embodiment, the effective dose of B. coagulans alleviates anxiety- and depression-symptoms, when compared to placebo control.

In one embodiment, method of treating depression symptoms by B. coagulans Unique IS-2 encompasses the restructuring of microbiome gut-brain axis by treating with Bacillus coagulans Unique IS-2 probiotic. In one embodiment, this method encompasses treating gut dysbiosis in subjects or patients affected by anxiety and/or depressive syndrome by B. coagulans Unique IS-2, which helps increasing gut eubiosis.

In one embodiment, method of treatment of individuals with Bacillus coagulans Unique IS-2 probiotic alleviates the anxiety and depression in affected individuals/patients.

In one embodiment, the probiotic formulation used for the method described herein is administered orally.

In one embodiment, the probiotic formulation is in the form of a beverage, food, or beverage or food supplement.

In one embodiment, the probiotic formulation is administered in combination with other therapeutic or ameliorative agents for treating the symptoms of depression.

In one embodiment, probiotic formulation described herein and the at least one additional agent are administered as a single composition or formulation.

In one embodiment, the depressive disorder is major depressive disorder, dysthymic disorder, seasonal affective disorder, mood disorder, mixed anxiety-depressive disorder or bipolar disorder.

In one embodiment, the depression is mild depression, moderate depression, severe depression or postpartum depression.

In one embodiment, the method described herein is employed as an adjunct to one or more other treatments or therapies for depression, anxiety or depressive or anxiety-related disorders.

In one embodiment, administration of the probiotic formulation comprising B. coagulans Unique IS-2 leads to decrease in any one or more of the symptoms associated with stress and anxiety in a subject/patient diagnosed with depressive disorder.

In one embodiment, administration of effective dosage of the probiotic formulation comprising B. coagulans Unique IS-2 leads to decrease of anhedonia in subjects treated with the formulation.

In one embodiment, administration of effective dosage of the probiotic formulation comprising B. coagulans Unique IS-2 leads to amelioration of one or more of somatic symptoms associated with anxiety, such as gastrointestinal disorders (IBS), back pain, chest pain, shortness of breath, heart palpitations, problems with sleep or appetite, and fatigue.

In one embodiment, administration of effective dosage of the probiotic formulation comprising B. coagulans Unique IS-2 leads to amelioration of one or more of symptoms associated with irritability, mood swings, depressed mood, disturbed sleep, listlessness, cognitive changes, short term memory loss, anxiousness, restlessness, tension, and anhedonia.

In one embodiment, administration of the probiotic formulation comprising B. coagulans Unique IS-2 leads to decrease in immobility associated with depressive syndrome or anxiety in subjects treated with the formulation.

In one embodiment, treatment with the probiotic formulation comprising B. coagulans Unique IS-2 leads to normalization of the increased plasma levels of L-tryptophan, L-kynurenine, kynurenic acid, and 3-hydroxyanthranilic acid found associated with depression and anxiety.

In one embodiment, treatment with B. coagulans Unique IS-2 restores the villi/crypt ratio restored to normal level after treatment.

In one embodiment, six weeks of Bacillus coagulans Unique IS-2 treatment also normalizes the number of goblet cell count in anxiety and depression affected individuals.

In one embodiment, Bacillus coagulans Unique IS-2 treatment positively impacts the intestinal epithelium and integrity.

In one embodiment, treatment with B. coagulans Unique IS-2 probiotic formulation also leads to normalization/reversal of the shift from the synthesis of 5-HT from tryptophan to the kynurenine pathway in stressed individuals, which leads to the accumulation of neurotoxic metabolites like 3-hydroxy anthranilic acid,

In one embodiment, treatment with Bacillus coagulans Unique IS-2 leads to restoration of the intestinal permeability associated with healthy individuals. In one embodiment, the treatment leads to a restoration of levels of tight junction proteins in people with stress or anxiety, and restoration of the levels of goblet cells and tight junction proteins which are reduced in stressed, depressed and anxious subjects. This impact in affected individuals is correlated with the reduction of goblet cells and TJs proteins such as occludin, ZO-1 in the colonic mucosa. Gut histology altered by depression, stress and anxiety is restored well by Bacillus coagulans Unique IS-2 treatment.

In one embodiment, the B. coagulans Unique IS-2 treatment leads to an increase in villus to crypt depth ratio and goblet cell count.

Healthy intestinal microbiota and their metabolites play an important role in maintaining the gut ecosystem in the host. These findings provide new insight into the complex interplay between the CNS and intestine in the therapeutic effects of probiotics for stress-related disorders.

Moreover, reshaping the intestinal microbial ecosystem by probiotic ingestion positively affects mood behaviour. Short chain fatty acids (SCFAs) such as acetate, butyrate, and propionate are the microbial metabolites by-products that can enter into the systemic circulation, cross the blood-brain barrier, and modulate the neural circuitry in the brain. The reduced levels of SCFA were found to be reduced in the macaques depression model and in depressive patients. Moreover, decreased levels of acetic acid and propionic acid are also reported in the chronic stress model of mice and depressive patients.

In one embodiment, treatment with Bacillus coagulans Unique IS-2 can enhance the levels of SCFA.

In one embodiment, the alleviation of symptoms associated with depression and/or anxiety following Bacillus coagulans Unique IS-2 treatment is due to reshaping the microbiota-gut-brain axis.

The present disclosure also provides methods of treating a subject in need thereof comprising administering to a subject a therapeutic composition comprising GABA-producing bacteria. As set forth herein, the GABA-producing bacteria can produce GABA in the subject's gut. The GABA can diffuse into other systems of the subject's body (e.g., the circulatory and nervous systems). There, the endogenous GABA can act as a neurotransmitter. In some embodiments, increased levels of GABA (e.g., in the nervous system) can help to alleviate the symptoms of depressive disorder or anxiety.

In one embodiment, the B. coagulans treatment leads to reversal of changes in neurological chemicals/transmitters; such as BDNF, serotonin, C-reactive protein, IL-1β, TNF-α and dopamine. In one embodiment, treatment with the probiotic formulation comprising at least 2 billion cfu of B. coagulans Unique IS-2 leads to reversal of decreased levels of BDNF and serotonin and increased levels of C-reactive protein, TNF-α, and dopamine, that are associated with stress.

Pharmaceutical preparations comprising the multistrain probiotic formulations disclosed herein may be in the form of solids such as tablets, granules, powders, capsules, and liquids such as solutions, emulsions, suspension and the like.

Pharmaceutically acceptable or appropriate carriers for use in the pharmaceutical preparations comprising the multistrain probiotic formulations disclosed herein can be, but are not limited to, organic or inorganic, solid or liquid excipients which is suitable for the selected mode of application such as oral application or injection, and administered in the form of a conventional pharmaceutical preparation. Examples of carriers or excipients in the preparation include, but are not limited to starch, lactose, glucose, sucrose, dextrin, cellulose, paraffin, fatty acid glyceride, water, alcohol, gum arabic and the like. If necessary, auxiliary, stabilizer, emulsifier, lubricant, binder, pH adjustor controller, isotonic agent and other conventional additives may be added.

The current invention also encompasses methods for making and using this formulation, and also encompasses compositions comprising this formulation and carriers, additives, synbiotics or any other components required for administration of this formulation.

### EXAMPLES

### Example 1: Materials and Study Design

### Microbial Strain:

Bacillus coagulans (B. coagulans) Unique IS-2 has been isolated by Unique Biotech Ltd, India. It is well characterized and has been deposited in MTCC (Microbial Type Culture Collection), India under Indian Patent deposit with the number MTCC 5260 (Bacillus coagulans Unique IS-2). B. coagulans Unique IS-2 has also been deposited in ATCC, and has the accession number B. coagulans Unique IS-2. B. coagulans Unique IS-2 exhibits probiotic properties and being a spore forming probiotic, is stable over a wide range of temperatures with potential applications in a variety of formulations and foods (Ref 11 &12, Madempudi, et al & Ahire et al).

A deposit of the Bacillus coagulans (B. coagulans) Unique IS-2 microbial strain has been made in a depository affording permanence of the deposit and ready accessibility thereto by the public if a patent is granted. The strain with its corresponding accession numbers is given below:

Bacillus coagulans Unique IS-2: MTCC 5260 (ATCC PTA-11748)

The Microbial Type Culture Collection and Gene Bank (MTCC facility in IMTECH, Sector −39, Chandigarh, India—160036), is an affiliate member of the World Federation for Culture Collections (WFCC) and is registered with the World Data Centre for Microorganisms (WDCM).

### Animals

Pregnant female Sprague-Dawley (SD) rats with a body weight of 200-250 g were purchased and allowed to deliver pups in the animal house facility of NIPER, Hyderabad, India. Both male and female SD rats were employed for this study. Except during the period of MS, pups were kept along with their respective mothers until 21 days of weaning time. Standard housing conditions like 12/12 h light/dark cycle, the controlled temperature of 24±2° C., and humidity were maintained throughout the study. Rats also had free access to food and water ad libitum. The protocols and methods of animal experiments were approved by the Institutional Animal Ethics Committee of the National Institute of Pharmaceutical Education and Research (NIPER), Hyderabad, India.

### Study Design for MS and CUMS

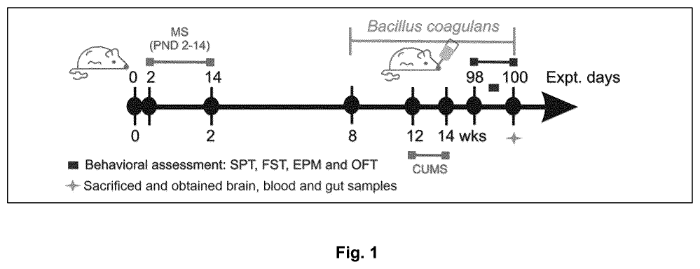

As depicted in FIG. 1 and published in Ref 13, Satti et al, (incorporated herein in its entirety) at an early age rats were subjected to MS and then exposed to CUMS for 2 weeks at the age of 12 weeks to mimic the early- and late-life stress. During MS, pups were separated from their respective mother dams and kept in a different cage with sterile shredded paper bedding for 3 h per day from postnatal day 2 (PND2) through PND14. Post-weaning male and female rats were separately group-housed (3-4 per cage). The CUMS was given between 12th week to 14th week of the study to mimic the late-life stress. The CUMS procedure is already described in our previous study (Ref 13, Satti et al). At the end of stress-exposure, the neurobehavioral assessments were performed using SPT, FST, EPM, and OFT assays. Post-behavioral studies, rats were sacrificed and blood, frontal cortex, hippocampus, and intestine samples were collected and stored at −80° C. till further analysis.

### Experimental Groups and Probiotic Regime

Male and female rats were randomized into 3 study groups (n=6-8 animals per group): [1] Vehicle control; [2] Vehicle+Stress (MS+CUMS) and [3] Probiotic+Stress (MS+CUMS). The preformulated probiotic formulation contained 2 billion CFU of Bacillus coagulans Unique IS-2 per dosage. The vehicle/probiotic treatment was initiated in rats aged 7-8 weeks and continued for the next 6 weeks. Six weeks of probiotic treatment was found to be effective for anxiety- and depression-like behaviors in rats (Ref 13, Satti et al). The probiotic formulation was freshly prepared each day before administration and provided daily between 4-6 pm. The probiotic-containing lyophilized powder was dissolved in drinking water and given to Probiotic+Stress (MS+CUMS) group. The vehicle control rats were fed with a carrier matrix. The body weights of rats were recorded weekly once throughout the study.

### Statistical Analysis

The data were analyzed by one-way analysis of variance (ANOVA) followed by post-hoc Dunnett's multiple comparisons test. The sex-specific correlation was determined using two-way ANOVA followed by Bonferroni's multiple comparisons test. The values p<0.05 were considered statistically significant. Data are tabulated and plotted as mean±S.E.M.

### Example 2: Behavioral Assessment for Anxiety- and Depression-Like Behavior

### 2.1. Sucrose Preference Test (SPT)

The anhedonia-like phenotype was assessed employing SPT in rats, as described in Ref 13, Satti et al. Briefly, in this test, on day 1, rats were allowed to drink ad libitum 1% w/v sucrose solution given through two drinking bottles. On the second day, one bottle of sucrose and another regular drinking water (200 mL) was provided, and to negate the effects of side-preference, the bottle position was exchanged every 6 h. Afterwards, rats were subjected to food and water deprivation for 12 h. On the test day (day 3), the same procedure was implemented as day 2. The consumption of drinking water, sucrose solution, and overall consumption was recorded after 12 h. The % sucrose preference was calculated as: % Sucrose consumption=[amount of sucrose solution consumed in 12 h/total amount of sucrose solution and water consumed in 12 h]×100.

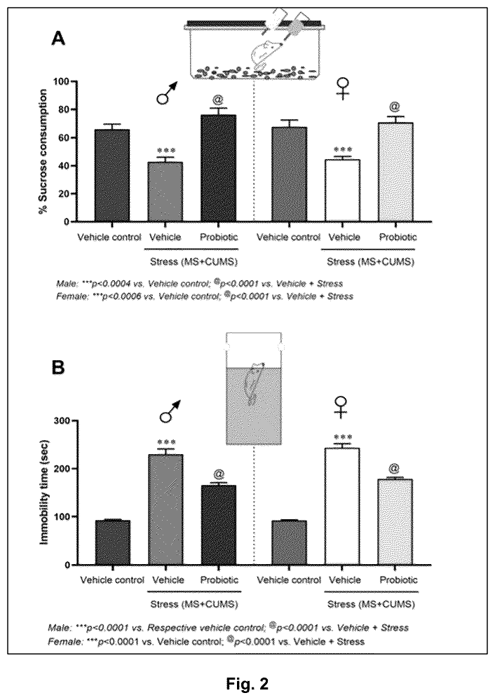

It was observed that Bacillus Coagulans Unique IS-2 increased sucrose consumption in the SPT. The application of MS+CUMS significantly decreased the sucrose consumption in both male (p<0.0004) and female rats (p<0.0006) compared to vehicle control animals. Six weeks of treatment with Bacillus Coagulans Unique IS-2 normalized the anhedonia-like phenotypes as indicated by the significant increase in sucrose preference in both male and female rats (p<0.0001 in both cases) vs. respective MS+CUMS exposed rats (FIG. 2A). No sex-specific interaction was noted after application of two-way ANOVA followed by Bonferroni's multiple comparisons test [F(1,48=0.04473, p=0.8334].

### 2.2. Forced Swimming Test (FST)

FST was used to assess depression-like phenotype in rats. The method has already been described in Ref 13 & 14, Satti et al & Dandekar et al, and has also been used to examine the effects of prebiotics and probiotics on depression-like behavior (Ref 9, Liu et al). On day 1 of the test, rats were immersed in a cylindrical glass tank (50 cm height×25 cm diameter) containing 30 cm of water with a temperature of 25° C. and allowed to swim for 15 min. After 24 h, each rat was re-exposed to the same glass tank. The immobility time was recorded by an observer for a test period of 5 min, who was blind to the treatment condition.

It was observed that Bacillus Coagulans Unique IS-2 increased immobility time in FST. Rats exposed to MS+CUMS displayed a significant increase in immobility time in both male and female rats (p<0.0001 in both) compared to a vehicle control group. Bacillus Coagulans Unique IS-2 treatment significantly decreased the immobility time in male and female animals (p<0.0001 in both) as compared to that in MS+CUMS group (FIG. 2B).

### 2.3. Elevated Plus-Maze Test (EPM)

EPM is one of the most frequently used rodent tests for assessing anxiety-like behavior and is well described in Ref 14, Dandekar et al. The apparatus consists of two opposing open arms (50 cm×10 cm), two opposing enclosed arms (50 cm×10 cm×40 cm), and is elevated to 50 cm height. During the test, each rat was placed on the center platform facing towards one of the open arms. The number of entries in open and closed arms and time spent in each arm were recorded for 5 min by an observer blind to the treatment groups. After each animal, the maze was wiped with 30% isopropanol to remove the cue of the earlier animal.

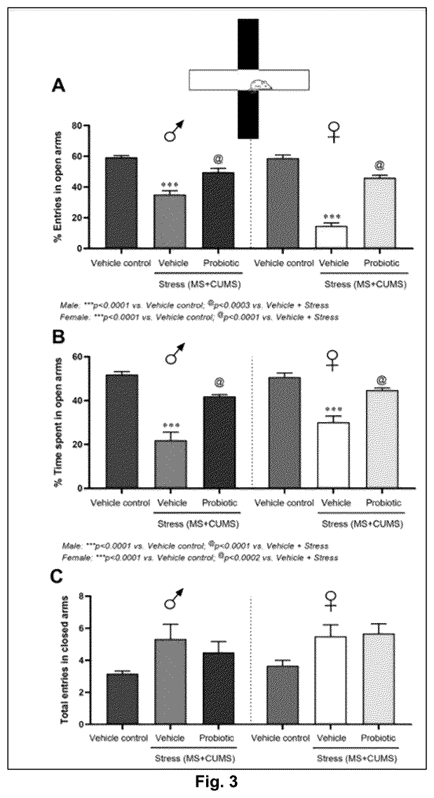

It was observed that Bacillus Coagulans Unique IS-2 increased open arm activity in EPM test. The application of MS+CUMS in both male and female rats showed a significant decrease in the number of entries (p<0.0001 in both cases, FIG. 3A) and time spent in open arms (p<0.0001 in both cases, FIG. 3B) as compared to the vehicle-treated group. The administration of Bacillus Coagulans Unique IS-2 probiotic reversed this state significantly in both sexes. The probiotic treatment significantly reversed the effect of MS+CUMS, as percentage entries (p<0.0003 and p<0.0001 in male and female rats, respectively) and percentage time spent in the opened arms increased significantly in both male (p<0.0001) and female (p<0.0002) groups compared to vehicle control. The total number of closed arms entries did not affect (p>0.05) across the groups (FIG. 3C).

### 2.4. Open Field Testing (OFT)

The open field apparatus consisted of a 50×50 cm fiber box, and the animals were put in the center field to let them explore for 5 mins. At the end of each test, the apparatus was wiped with 70% v/v alcohol solution to avoid any odor cue. The total distance travelled by the animal was analyzed by the ANY-maze® software (Stoelting Co., United Kingdom). Also, Bacillus Coagulans Unique IS-2 did not affect locomotor activity.

The total ambulatory activity was recorded using ANY-maze® software. The horizontal activity of rats was remained unchanged (p>0.05) across the treatment groups. Hence, the application of MS+CUMS effectively developed anxiety- and depression-like behaviors in rats. The stress-exposed rats also displayed an anhedonic-like phenotype in the SPT, as indicated by less sucrose consumption. Six weeks of treatment with Bacillus coagulans Unique IS-2 normalized these neurobehavioral aberrations.

### Example 3: Quantification of CRP, TNF-α IL-1β and BDNF in Frontal Cortex and Hippocampus Using ELISA

The hippocampus and frontal cortex brain samples were processed as described in Ref 13 & 14, Satti et al & Dandekar et al. Protein estimation was done using the Rapid Gold BCA protein assay kit. The cytokines levels were estimated in the frontal cortex and hippocampus using commercially available rat CRP ELISA (DY1744), rat IL-1β ELISA (DY501-05) and TNF-α (DY510-05) kits from R&D Systems. The levels of BDNF were also estimated using the BDNF ELISA kit (R&D systems, DBNT00). Results were calculated based on the absorbance of complex antigen-antibodies.

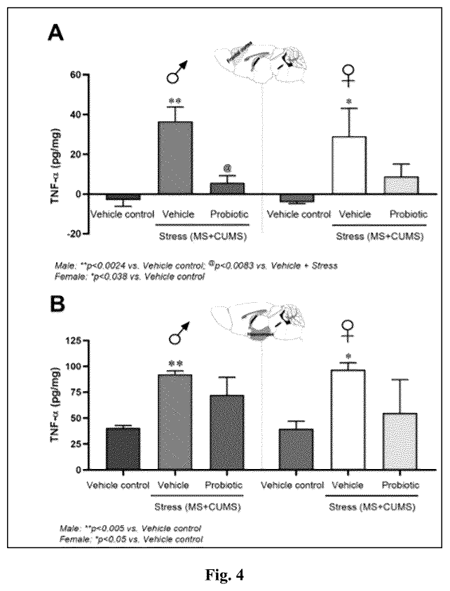

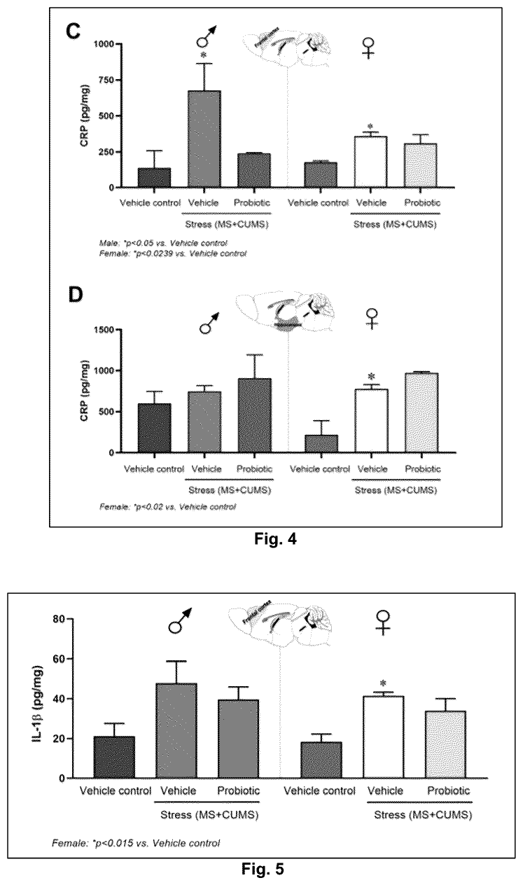

Using ELISA kits, we estimated the concentration of BDNF, TNF-α, and CRP cytokines in the frontal cortex and hippocampus (Table 1 and FIG. 4A-D). Application of MS+CUMS in rats showed a significant decrease in BDNF in the hippocampus (p<0.0219 vs. vehicle control in male rats) and a profound increase in the concentration of cytokines (TNF-α: p<0.005 and p<0.05 in male and female rats, respectively) and (CRP: p<0.02 in female rats) in the hippocampus. We also observed elevated levels of TNF-α (p<0.0024 and p<0.038 in male and female rats, respectively) and CRP (p<0.05 and p<0.0239 in male and female rats, respectively) and IL-1β (p<0.015 in female rats) in the frontal cortex region of MS+CUMS exposed rats (FIG. 5).

In this study, six-week administration of Bacillus coagulans Unique IS2 restored the decreased levels of BDNF in the hippocampus (Table 1). However, we did not observe a noticeable change in the BDNF expression in the frontal cortex of both male and female rats. The elevated levels of TNF-α in the frontal cortex of MS+CUMS group was significantly reversed (p<0.0083 in male rats) following Bacillus coagulans Unique IS-2 treatment as compared to the vehicle+stress (MS+CUMS) group (FIGS. 4A and B). We also observed reduced levels of CRP and IL-1β in the frontal cortex of Bacillus coagulans Unique IS-2 administered male rats compared to the vehicle+stress (MS+CUMS) group.

We observed elevated levels of TNF-α, IL-1β and CRP in the hippocampus and frontal cortex after MS+CUMS exposure. In the present study, 6 weeks of treatment with Bacillus coagulans Unique IS-2 abrogated the elevated levels of CRP, IL-1ß and TNF-α cytokines in the hippocampus and/or frontal cortex.

TABLE 1
Impact of Bacillus Coagulans Unique IS-2 administration
on BDNF in the frontal cortex and hippocampus

| | | | | BDNF (pg/mg) in | |
|---|---|---|---|---|---|
| | | BDNF (pg/mg) in Frontal | | Hippocampus | |
| | | cortex (Mean ± S.E.M.) | | (Mean ± S.E.M.) | |
| SN | Treatment | Male | Female | Male | Female |
| 1 | Vehicle control | 271.8 ± 9.92 | 280.9 ± 37.24 | 510.1 ± 41.97 | 399.6 ± 74.64 |
| 2 | Vehicle + Stress | 239.0 ± 19.55 | 248.0 ± 44.10 | 318.2 ± 32.52* | 345.2 ± 21.45 |
| | (MS + CUMS) | | | | |
| 3 | Probiotics + Stress | 300.9 ± 15.34 | 254.0 ± 34.03 | 422.8 ± 42.47 | 442.3 ± 31.30 |
| | (MS + CUMS) | | | | |

### Example 4: Estimation of Doublecortin, GFAP, and Zona Occludens Levels in the Hippocampus Using an Immunoblotting Method

The hippocampal tissues were thawed and extracted protein using tissue protein extraction reagent (TPER) and protease inhibitor cocktail (Cat. No. ML051). The protein levels were estimated in the supernatant using a BCA rapid gold protein assay kit (Cat. No. A53225). The equal amount of protein was loaded on SDS PAGE, and then proteins were separated and transferred to the nitrocellulose membrane. The membrane was incubated in the 5% bovine serum albumin (BSA) solution for 60 min and then overnight in primary antibody (1:1000) [GFAP (Cat. No. SC-33673), doublecortin (Cat. No. SC-271390), zona occludens (Cat. No. SC-33725) or β-actin (Cat. No. SC-47778)]. The protein bands were developed by incubating the membrane in the respective secondary antibody (Cat. No. 31430) at 1:20,000 dilution, followed by clarity ECL substrate (Cat. No. 170-5061). Band intensities were calculated using ImageJ software.

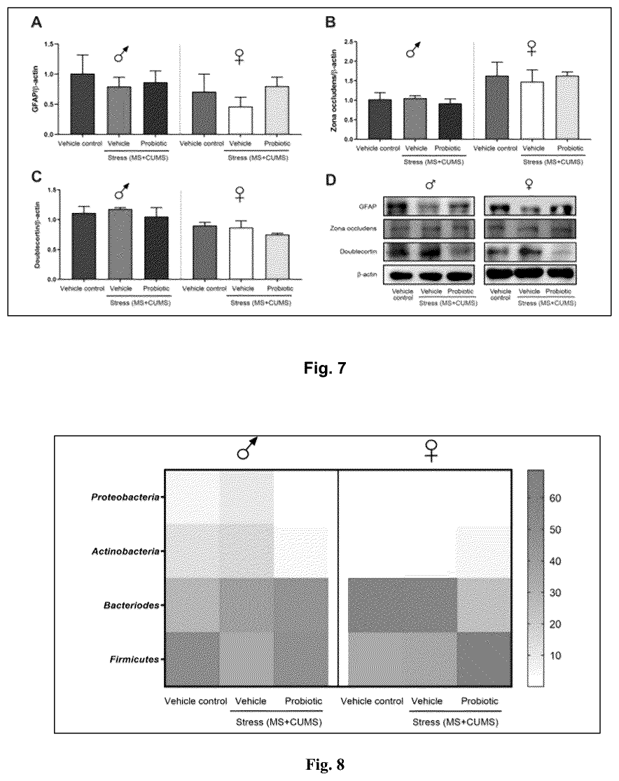

Changes in the hippocampal levels of GFAP, zona occludens, and doublecortin were estimated by an immunoblotting method. No considerable change was seen in MS+CUMS, and Bacillus coagulans Unique IS-2 treated rats compared to vehicle control animals (FIG. 7).

### Example 5: Quantification of DA, NA, and 5-HT Using HPLC-ECD

We estimated the levels of dopamine (DA), NA (noradrenaline or norepinephrine), and 5-HT (serotonin) neurotransmitters in the frontal cortex by high-performance liquid chromatography (HPLC) coupled with an electrochemical detector as described in Ref 13 & 14, Satti et al & Dandekar et al. Briefly, brain samples were homogenized in 0.2 M perchloric acid containing 0.2 mg/mL L-cysteine. Homogenates were centrifuged at 14,000 rpm, then supernatants were injected into the HPLC-ECD system [BASi Epsilon ECD detector (PK/2008/006)]. The calibration accuracy for DA, NA and 5-HT was 97.67-105%, 97.36-102.72%, and 97.76-105%, respectively. Data was analyzed using Agilent and EZChrom Elite 3.2.0.

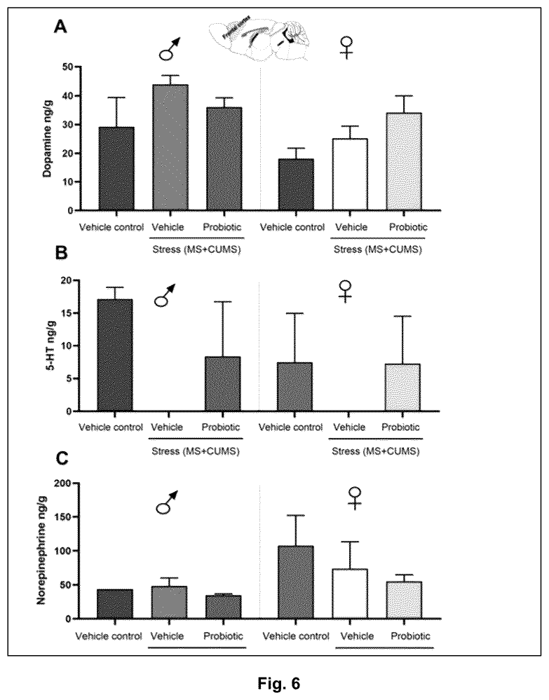

A noticeable decrease in the content of 5-HT and a marginal increase of DA was seen in the frontal cortex of both male and female stress-exposed (MS+CUMS) rats. These altered concentrations of 5-HT and DA were restored to the control level following Bacillus coagulans Unique IS-2 treatment (FIG. 6A-B). No noticeable change was seen in the levels of NA in the frontal cortex across the treatment groups (FIG. 6C).

Dopaminergic hyperfunctioning due to CUMS exposure was seen via increased levels of dopamine in the frontal cortex in both male and female vehicle groups compared to their respective vehicle control groups. Probiotic treatment showed sex-dependent effects, which could be observed as a decrease in dopamine levels in the case of males and an increase in dopamine in the case of female groups compared to their respective vehicle control groups. The levels of NA were high in the vehicle control group of females, which was decreased after CUMS induction in vehicle-treated animals, and probiotic treatment did not modify these levels. However, the levels of NA remained the same across the treatment group in male rats. Bacillus coagulans Unique IS-2 treatment restored the levels of 5-HT in both males and females. We observed reduced levels of BDNF in the hippocampus of CUMS+MS exposed male rats compared to female. As reported earlier, sex-specific changes in the hippocampal BDNF may be ascribed to the oestrogen (Ref 15, Franklin et al). BDNF levels were reduced by the induction of CUMS, which were recovered by the Bacillus coagulans Unique IS-2 treatment, thereby restoring the synaptic plasticity and survival and differentiation of 5-HT neurons. The effect of CUMS, a probiotic on neurotransmitters' levels was insignificant (p>0.05). The effect of CUMS and probiotic treatment was taken together in the case of dopamine and 5-HT but not on norepinephrine in both male and female groups.

### Example 6: Estimation of Tryptophan Metabolites Using LC-MS/MS

Chromatographic separation of L-tryptophan, L-kynurenine, kynurenic acid, 3-hydroxyanthranilic acid, and tolbutamide (IS) was achieved on Waters ACQUITY UPLC® BEH C18 column (2.1 mm×100 mm, 1.7 μm) at 30° ° C. A gradient mobile phase system [(0.1% formic acid (A) and acetonitrile (B)] with a flow rate of 0.2 mL/min was applied with a total run time of 15 min having a gradient elution (Tmin/Acetonitrile %): 0.0-0.0/2, 0.0-0.5/2, 0.5-2.0/20, 2.0-2.5/20, 2.5-3.0/35, 3.0-3.5/35, 3.5-4.0/55, 4.0-4.5/55, 4.5-5.0/2, 5.0-6.0/2, 6.0-8.0/98, 8.0-10.0/2, 10.0-15.0/2. The plasma sample (50 μL) was mixed with ACN (150 μL) and tolbutamide (100 ng/mL). The supernatant was collected by centrifugation at 10,000 rpm for 10 min. The processed samples were stored at 4° C. in an autosampler and an injection volume of 10 μL was used. A quadrupole time-of-flight (Q-TOF) mass spectrometer (Q-TOF LC/MS 6540 series, Agilent Technologies, USA) coupled with electrospray ionization (ESI) was used for mass spectrometric studies. The operating source conditions for L-tryptophan, L-kynurenine, kynurenic acid, 3-hydroxyanthranilic acid, and internal standard in positive ESI mode were optimized as described in Ref 13, Satti et al.

It was observed that Bacillus coagulans Unique IS-2 normalized the tryptophan metabolites.

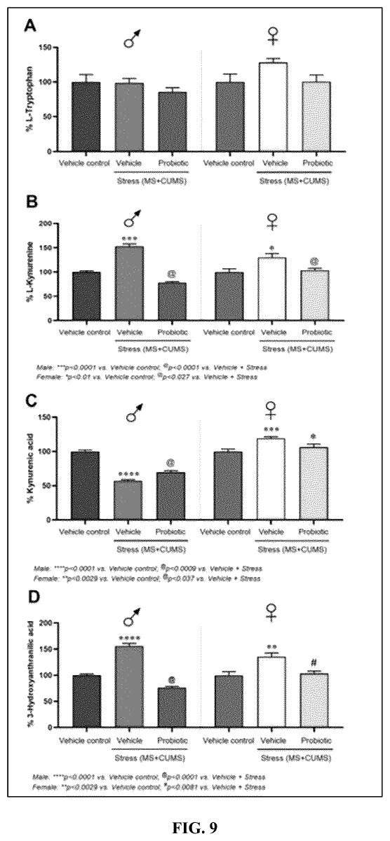

Application of MS+CUMS showed an increase in the plasma levels of L-tryptophan in female rats, L-kynurenine (p<0.0001 and p<0.01 in male and female rats, respectively), kynurenic acid (p<0.0029 in female rats), and 3-hydroxyanthranilic acid (p<0.0001 and p<0.0029 in male and female rats, respectively) as compared to that in vehicle-treated rats (FIG. 9A-D). However, a significant decrease in kynurenic acid (p<0.0001, FIG. 9C) and no change in the L-tryptophan (p>0.05, FIG. 9A) were observed in MS+CUMS exposed male rats. Interestingly, the altered levels of these metabolites were normalized by 6 weeks in Bacillus coagulans Unique IS-2 treated male and female rats. Two-way ANOVA showed a significant interaction between variables probiotic treatment and sex of animals, L-kynurenine [F(2,30)=10.85, p=0.0003], kynurenic acid [F(2,30)=58.89, p<0.0001] and 3-hydroxyanthranilic acid [F(2,30)=10.83, p=0.0003].

The synthesis of 5-HT from tryptophan shifted to the kynurenine pathway, which led to the accumulation of neurotoxic metabolites like 3-hydroxy anthranilic acid, which is normalized by Bacillus coagulans Unique IS-2 treatment.

### Example 7: Analysis of Bacterial Abundance by RT-qPCR

The method of DNA extraction and RT-qPCR procedure has been described in Ref 13 & 14, Satti et al & Dandekar et al. The fecal DNA was extracted using QIAamp Power Fecal Pro DNA Kit (Cat. No. 51804). After quality and quantity determination of DNA, samples were processed for real-time qPCR using QuantStudio™ 7 Pro Real-Time PCR System (Applied Biosystems). Amplification and detection were performed using iTaq™ Universal SYBR Green Supermix (Biorad) (Cat. No. 172-5122). The details of primers have been described in earlier study 21. The thermal cycling protocol is set with initial denaturation at 95° C. for 15 s, annealing at 60° C. for 30 s, and extension at 72° ° C. for 30 s, followed by 35 cycles of 95° C. with 0.5° C. increment at 2-5 sec/step. The bacterial abundance of Firmicutes, Bacteroidetes, Actinobacteria, Proteobacteria was calculated with respect to 16S ribosomal RNA as per in Ref 13, Satti et al. The percentage abundance was calculated as (each bacterial population in fecal samples)/(each bacterial population in the highest one)×100.

The Firmicutes and Bacteroidetes were prominently appeared in the rat's fecal samples (FIG. 8). The decrease in Firmicutes to Bacteroidetes ratio was noted in rats exposed MS+CUMS compared to normal animals. At the same time, we did not see any change in female rats (Table 2). Administration of Bacillus coagulans Unique IS-2 increased the Firmicutes to Bacteroidetes ratio in female rats compared to respective MS+CUMS group.

TABLE 2
Firmicutes to Bacteroidetes (F/B) ratio:

| | | Vehicle + | Probiotic + |
|---|---|---|---|
| | Vehicle | Stress | Stress |
| Treatment | control | (MS + CUMS) | (MS + CUMS) |
| Male (F/B ± S.E.M.) | 1.84 ± 0.62 | 0.85 ± 0.43 | 1.04 ± 0.05 |
| Female (F/B ± S.E.M.) | 0.54 ± 0.05 | 0.58 ± 0.13 | 1.15 ± 0.17 |

### Example 8: Quantification of SCFA by Gas Chromatography (GC) with Flame-Ionization Detection (FID)

The levels of acetate, butyrate, and propionate in rats' fecal samples were quantified using the GC-FID method. The detailed procedure of GC-FID has already been described in Ref 14, Dandekar et al. Briefly, 100 mg of fecal samples were mixed with 1 mL of water and homogenized at 6500 rpm for 30 s and then stored for 30 min at 4° C. Later we obtained the supernatant and mixed it with HCl. Then, extracted was done using diethyl ether (DE). The DE extract (1 mL) was transferred into a glass insert in a GC vial and capped tightly after adding 5 μL of BSTFA (A5603) and vortexed for 5 sec. The mixture was kept in the GC vial at 37° C. as described earlier in Ref 16, Zhang et al, the derivatized samples were loaded to GC-FID (8890, Agilent Technologies). Finally, concentration of acetate, propionate, and butyrate was determined by taking a reference of known amount of SCFAs. As shown in Table 3, lower levels of propionate and butyrate were seen in the MS+CUMS group. Treatment with Bacillus coagulans Unique IS-2 increases the levels of acetate, propionate and butyrate when compared to (MS+CUMS) group.

Reshaping the intestinal microbial ecosystem by probiotic ingestion positively affected mood behavior. SCFAs such as acetate, butyrate, and propionate are the microbial metabolites byproducts that can enter into the systemic circulation, cross the blood-brain barrier, and modulate the neural circuitry in the brain (Ref 17, Frost et al).

Considering the association of SCFAs with depressive behavior, we estimated the acetate, butyrate, and propionate levels in the fecal samples of both male and female rats. The acetate, butyrate, and propionate levels were reduced in the MS+CUMS group and increased significantly in the Bacillus coagulans IS2 group. Recent study indicates that the beneficial effects of microbiome-derived butyrate may be mediated via modification of mitochondrial functions (Ref 10, Anderson et al). Considering the influence of gut micro biome on the alteration of mitochondrial functions, anxiolytic- and antidepressant-like effects of Bacillus coagulans Unique IS-2 may be partly affecting the metabolic activity. Based on our results, we suggest that Bacillus coagulans IS2 can enhance the levels of SCFAs that subsequently activate the mood elevating processes.

TABLE 3
Effect of B.coagulans Unique IS-2 administration on SCFAs levels
(acetate, propionate, and butyrate) in the fecal samples

| | | Acetate | | Propionate | | Butyrate | |
|---|---|---|---|---|---|---|---|
| | | (Mean ± S.E.M.) | | (Mean ± S.E.M.) | | (Mean S.E.M.) | |
| S. No | Treatment | Male | Female | Male | Female | Male | Female |
| 1 | Vehicle | 29.55 ± 7.26 | 28.53 ± 2.70 | 11.32 ± 3.39 | 10.71 ± 2.15 | 5.93 ± 0.88 | 3.31 ± 0.69 |
| | control | | | | | | |
| 2 | Vehicle + | 39.32 ± 1.74 | 34.60 ± 6.07 | 9.27 ± 1.92 | 8.04 ± 1.96 | 2.10 ± 0.16^{#} | 2.96 ± 0.66 |
| | Stress | | | | | | |
| | (MS + CUMS) | | | | | | |
| 3 | Probiotics + | 42.53 ± 7.254 | 46.78 ± 11.2 | 12.45 ± 1.86 | 15.87 ± 5.31 | 2.657 ± 1.469 | 1.54 ± 0.62 |
| | Stress | | | | | | |
| | (MS + CUMS) | | | | | | |

### Example 9: Immunohistochemistry and H&E Staining

The distal part of the colon was fixed in 4% paraformaldehyde. Using a microtome, 5 μm thick coronal sections were taken of the paraffin embedded colons. The mounted slides are deparaffinized and dehydrated using xylene, followed by different dilutions of ethanol. Citrate-based antigen retrieval was performed. Further steps of blocking and staining are done using the Vectastain Elite ABC Universal PLUS kit (PK-8200). Occludin (SC-133256) was used as the primary antibody at 1:100 dilution. The quantification of occludin-positive immunoreactivity was done using ImageJ software and represented as mean optical density (MOD). H&E staining was employed to assess the microscopic morphological changes such as villus height, crypt depth ratio, and goblet cell count.

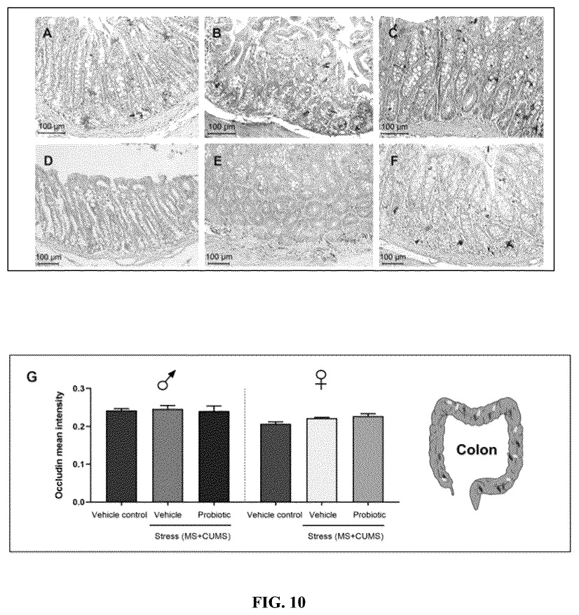

The mean optical density of the occludin-positive area was estimated. No significant change (p>0.05) was seen in the occludin-positive area in MS+CUMS exposed animals as well as Bacillus coagulans Unique IS-2 treated rats (FIG. 10A-G).

Bacillus coagulans Unique IS-2 normalized the villi height, crypt depth, and goblet cell count. A significant decrease in the villi/crypt ratio (p<0.0023 and p<0.0001 in male and female rats, respectively) and the number of goblet cells in both male (p<0.02) and female animals (p<0.005) was observed in MS+CUMS recipient animals compared to the vehicle-treated group. The villi/crypt ratio restored to normal level after treatment with Bacillus coagulans Unique IS in male [p<0.0019 vs. stress group (MS+CUMS)] and female animals [p<0.0001 vs. stress group (MS+CUMS)]. Six weeks of Bacillus coagulans Unique IS treatment also normalized the number of goblet cell count in both male (p<0.0001) and female (p<0.0009) rats compared to MS+CUMS groups. These findings indicate that Bacillus coagulans Unique IS-2 may positively impact the intestinal epithelium and integrity.

### REFERENCES

1. Capuco, A, Urits, I., Hasoon, J., Chun, R., Gerald, B., Wang, J. K., Kassem, H., Ngo, A. L., Abd-Elsayed, A., Simopoulos, T., Kaye, A. D., Viswanath, O. (2020) Current Perspectives on Gut Microbiome Dysbiosis and Depression. Adv Ther. 37(4):1328-1346.
2. Zheng, P., Zeng, B., Zhou, C., Liu, M., Fang, Z., Xu, X., Zeng, L., Chen, J., Fan, S., Du, X., Zhang, X., Yang, D., Yang, Y., Meng, H., Li, W., Melgiri, N. D., Licinio, J., Wei, H., Xie, P. (2016) Gut microbiome remodeling induces depressive-like behaviors through a pathway mediated by the host's metabolism. Mol Psychiatry. 21(6):786-96.
3. Jiang, H., Ling, Z., Zhang, Y., Mao, H., Ma, Z., Yin, Y., Wang, W., Tang, W., Tan, Z., Shi, J., Li, L., Ruan, B. (2015) Altered fecal microbiota composition in patients with major depressive disorder. Brain Behav Immun. 48:186-94.
4. Keller, D., Verbruggen, S., Cash, H., Farmer, S., Venema, K. (2019) Spores of Bacillus coagulans GBI-30, 6086 show high germination, survival, and enzyme activity in a dynamic, computer-controlled in vitro model of the gastrointestinal tract. Benef Microbes. 10(1):77-87.
5. Cao, J., Yu, Z., Liu, W., Zhao, J., Zhang, H., Zhai., Chen, w. (2020) Probiotic characteristics of Bacillus coagulans and associated implications for human health and diseases. JFF. 64: 103643.
6. Iordache, M. M., Tocia, C., Aschie, M., Dumitru, A., Manea, M., Cozaru, G. C., Petcu, L., Vlad, S. E., Dumitru, E., Chisoi, A. (2022) Intestinal Permeability and Depression in Patients with Inflammatory Bowel Disease. J Clin Med. 11(17):5121.
7. Liu, R. T., Rowan-Nash, A. D., Sheehan, A. E., Walsh, R. F. L., Sanzari, C. M., Korry, B. J., Belenky, P. (2020) Reductions in anti-inflammatory gut bacteria are associated with depression in a sample of young adults. Brain Behav Immun. 88:308-324.
8. Liu, R. T., Walsh, R. F. L., Sheehan, A. E. (2019) Prebiotics and probiotics for depression and anxiety: A systematic review and meta-analysis of controlled clinical trials. Neurosci Biobehav Rev. 102:13-23.
9. Chen, L. M., Bao, C. H., Wu, Y., et al. (2021) Tryptophan-kynurenine metabolism: a link between the gut and brain for depression in inflammatory bowel disease. J Neuroinflammation. 18:135.
10. Anderson, K. E., Ricigliano, V. A., Copeland, D. C., Mott, B. M., Maes, P. (2023) Social Interaction is Unnecessary for Hindgut Microbiome Transmission in Honey Bees: The Effect of Diet and Social Exposure on Tissue-Specific Microbiome Assembly. Microb Ecol. 85(4): 1498-1513.
11. Madempudi, R. S., Neelamraju, J., Ahire, J. J., Gupta, S. K., Shukla, V. K. (2020) Bacillus coagulans Unique IS2 in Constipation: A Double-Blind, Placebo-Controlled Study. Probiotics Antimicrob Proteins. 12(2):335-342.
12. Ahire, J. J., Neelamraju, J., Madempudi, R. S. (2020) Behavior of Bacillus coagulans Unique IS2 spores during passage through the simulator of human intestinal microbial ecosystem (SHIME) model. LWT: 124.
13. Satti, S., Palepu, M. S. K., Singh, A. A., Jaiswal, Y., Dash, S. P., Gajula, S. N. R., Chaganti, S., Samanthula, G., Sonti, R., Dandekar, M. P. (2023) Anxiolytic- and antidepressant-like effects of Bacillus coagulans Unique IS-2 mediate via reshaping of microbiome gut-brain axis in rats. Neurochem Int. 163:105483.
14. Dandekar, M. P., Palepu, M. S. K., Satti, S., Jaiswal, Y., Singh, A. A., Dash, S. P., Gajula, S. N. R., Sonti, R. (2022) Multi-strain Probiotic Formulation Reverses Maternal Separation and Chronic Unpredictable Mild Stress-Generated Anxiety- and Depression-like Phenotypes by Modulating Gut Microbiome-Brain Activity in Rats. ACS Chem Neurosci. 13(13):1948-1965.
15. Franklin, T. B., Perrot-Sinal, T. S. (2006) Sex and ovarian steroids modulate brain-derived neurotrophic factor (BDNF) protein levels in rat hippocampus under stressful and non-stressful conditions. Psychoneuroendocrinology. 31(1):38-48.
16. Zhang, S., Wang, H., Zhu, M. J. (2018) A sensitive GC/MS detection method for analyzing microbial metabolites short chain fatty acids in fecal and serum samples. Talanta. 196:249-254.
17. Frost, G., Sleeth, M. L, Sahuri-Arisoylu, M., Lizarbe, B., Cerdan, S., Brody, L., Anastasovska, J., Ghourab, S., Hankir, M., Zhang, S., Carling, D., Swann, J. R., Gibson, G., Viardot, A., Morrison, D., Louise Thomas, E., Bell, J. D. (2014) The short-chain fatty acid acetate reduces appetite via a central homeostatic mechanism. Nat Commun. 5:3611.

We claim:

1. A method of treating, preventing or ameliorating at least one symptom of anxiety and/or depression in a subject, the method comprising the step of administering to the subject an effective dosage of a probiotic formulation comprising the bacterial strain Bacillus coagulans.

2. The method of claim 1, wherein the B. coagulans in the probiotic formulation is the strain Bacillus coagulans Unique IS-2 (MTCC 5260).

3. The method of claim 1, wherein the effective dosage regimen of the probiotic composition is 1-2 doses a day administered every day for 4-8 weeks.

4. The method of claim 1, wherein the effective dosage regimen of the probiotic composition is 1-2 doses a day administered every day for six weeks.

5. The method of claim 3, wherein each dose of the probiotic composition comprises 1-10 billion cfu of Bacillus coagulans.

6. The method of claim 5, wherein each dose of the probiotic composition comprises 1-3 billion cfu of Bacillus coagulans.

7. The method of claim 1, wherein the administration of the effective dosage regimen of the probiotic formulation does not affect body weight or locomotor activity of the subject.

8. The method of claim 1, wherein the probiotic composition is administered to the patient as an adjunct to standard medical treatment (SMT) for anxiety and/or depression.

9. The method of claim 1, wherein the probiotic composition is administered orally to the patient.

10. The method of claim 1, wherein the probiotic formulation is administered to the subject in the form of a food product, a dietary supplement or a pharmaceutically acceptable composition.

11. The method of claim 1, wherein the probiotic formulation alleviates anxiety and depression symptoms by increasing gut eubiosis in the patient.

12. The method of claim 1, wherein administration of an effective dosage of the probiotic formulation comprising B. coagulans leads to amelioration of one or more symptoms associated with anxiety, such as gastrointestinal disorders (IBS), back pain, chest pain, shortness of breath, heart palpitations, problems with sleep or appetite, and fatigue.

13. The method of claim 1, wherein administration of the effective dosage of the probiotic formulation comprising B. coagulans IS2 leads to amelioration of one or more of symptoms associated with anxiety or depression such as irritability, mood swings, depressed mood, disturbed sleep, listlessness, cognitive changes, short term memory loss, anxiousness, restlessness, tension, and anhedonia.

14. The method of claim 1, wherein administration of an effective dosage of the probiotic formulation comprising B. coagulans leads to decrease of the depression symptom of anhedonia in subjects treated with the formulation.

15. The method of claim 1, wherein the administration of the effective dosage regimen of probiotic formulation alleviates anxiety and depression symptoms by modulating levels of tryptophan metabolites.

16. The method of claim 15, wherein the administration of the effective dosage regimen of probiotic formulation ameliorates symptoms of anxiety and/or depression by restoring the elevated levels of L-tryptophan, L-kynurenine, kynurenic acid, and 3-hydroxyanthranilic acid in the circulatory system of the subject.

17. The method of claim 1, wherein the administration of the effective dosage regimen of probiotic formulation ameliorates the symptoms associated with anxiety and/or depression by reversing the accumulation of neurotoxic metabolites associated with anxiety or depression in the subject.

18. The method of claim 17, wherein the neurotoxic metabolite is 3-hydroxy anthranilic acid.

19. The method of claim 1, wherein the administration of the effective dosage regimen of probiotic formulation ameliorates symptoms of anxiety and/or depression by reversing the decrease in levels of short chain fatty acids associated with anxiety or depression in the subject.

20. The method of claim 19, wherein the short chain fatty acids are acetate, propionate and/or butyrate.

21. The method of claim 1, wherein the administration of an effective dosage regimen of the probiotic formulation ameliorates symptoms of anxiety and/or depression in the subject by decreasing levels of proinflammatory cytokines in the subject.

22. The method of claim 1, wherein administration of an effective dosage regimen of the probiotic formulation ameliorates symptoms of anxiety and/or depression by reversing the elevated expression levels of IL-1β, TNF-α and C-reactive protein associated with anxiety and depression in the subject.

23. The method of claim 1, wherein the administration of an effective dosage regimen of the probiotic formulation ameliorates symptoms of anxiety and/or depression in the subject by increasing the number of goblet cells and improved crypt-to-villi ratio in the colon of the subject.

24. The method of claim 1, wherein administration of an effective dosage regimen of the probiotic formulation ameliorates symptoms of anxiety and/or depression by reversing the decrease in BDNF levels in subjects showing symptoms of anxiety and/or depression.

25. The method of claim 1, wherein administration of an effective dosage regimen of the probiotic formulation ameliorates symptoms of anxiety and/or depression by reversing the decrease in Firmicutes to Bacteroidetes ratio in subjects showing symptoms of depression and/or anxiety.
